(19) United States
(12) Patent Application Publication
(10) Pub. No.: US 2017/0135979 A1
(43) Pub. Date: May 18, 2017

(54) PDE-DELTA INHIBITOR FOR THE TREATMENT OF CANCER

(71) Applicant: Macau University of Science and Technology, Taipa (CN)

(72) Inventors: Xiao Jun Yao, Taipa (CN); Lai Han Leung, Taipa (CN); Lian Xiang Luo, Taipa (CN); Liang Liu, Taipa (CN)

(21) Appl. No.: 14/940,406

(22) Filed: Nov. 13, 2015

(51) Int. Cl. A61K 31/343

(52) U.S. Cl. CPC A61K 31/343

(57) ABSTRACT

The present invention relates to the administration of a novel compound advantageously efficacious as PDEδ inhibitor and its effects on subjects with cancer. More specifically, the present invention is directed to a method for administering a compound having favorable geometric properties for interacting with the PDEδ prenyl-binding pocket, namely has certain structural components such as a three-cyclic backbone and at least one benzoyl-moiety in a side chain having at least two substituents containing highly electronegative atoms and being linked to the backbone via an aliphatic chain, for treating a subject suffering from a disease such as cancer, in particular non-small-cell lung cancer. The presence of said structural components particularly contributes to an advantageous interaction with PDEδ, in particular with amino acids deep in the binding pocket. The present invention further provides a method to target tumor cells harboring an RAS gene mutation as well as pharmaceutical compositions comprising said compound.

### TECHNICAL FIELD

The present invention relates to the administration of a compound efficacious as PDEδ (PDE-Delta) inhibitor and its effects on subjects with cancer. More specifically, the present invention is directed to a method for administering a compound having certain structural components for treating a subject suffering from a disease such as cancer, in particular non-small cell lung cancer adenocarcinoma. The present invention further provides a method for targeting tumor cells harboring an RAS gene mutation as well as pharmaceutical compositions comprising said compound.

### BACKGROUND OF THE INVENTION

RAS proteins belong to a family of membrane-associated 21-kDa guanosine triphosphate (GTP)-binding proteins by cycling between ‘off’ and ‘on’ conformations that are conferred by the binding of guanosine diphosphate (GDP) and GTP, respectively. Namely, they cycle between inactive GDP-bound and active GTP-bound forms, wherein interconversion between both forms is catalyzed, for example, by GTPase activating proteins (GAP).

RAS proteins are central mediators involved in a variety of intracellular signaling pathways critical for cell proliferation, survival, and differentiation of cells. Three different mammalian RAS proteins and encoding genes have been identified, namely K-RAS (with two splice variants K-RAS4A and K-RAS4B with K-RAS4B being the more abundant isoform), H-RAS, and N-RAS. All RAS isoforms are reported to share 82% to 90% overall sequence identity as well as sequence identity in all of the regions responsible for GDP/GTP-binding, but they exhibit different C-terminal variable regions (prenylated cysteine) that target them to different cellular compartments and are responsible for membrane association and cellular localization (Spiegel, J. et al., Nat Chem Biol., 2014, 10:613-622, Cox, A. D. et al., Nat Rev Drug Discov., 2014, 13:828-851). They all are farnesylated and H-RAS, N-RAS and K-RAS4A are additionally S-palmitoylated in their variable regions.

RAS proteins interact with and can activate several downstream effectors in particular including raf protein kinases and phosphoinositide 3-kinases (PI3K) involved in cell survival and proliferation. Downstream signaling pathways activated by RAS are, for example, the PI3K-AKT-mTOR pathway and the raf-MEK-ERK pathway (Wang, Y. et al., J Med Chem., 2013, 56:5219-5230, Acquaviva, J. et al., Mol Cancer Ther., 2012, 11:2633-2643). Said RAS signaling strongly depends on the correct intracellular localization of the RAS proteins.

RAS proteins have been reported to be involved in the pathogenesis of several cancers. In particular, several mutations within the RAS protein encoding genes are reported to result in permanently activated RAS signaling pathways. It is generally assumed that about 30% of all human cancers harbor activating RAS mutations while being often not responsive to established therapies, making such RAS mutations, thus, to one of the most common known genetic causes of cancer. In this context, K-RAS is considered for being the most frequent mutated isomer in various cancers such as colon cancer, lung cancer, pancreatic cancer, and hematologic malignancies (Wang, Y. et al., J Med Chem., 2013, 56:5219-5230, Spiegel, J. et al., Nat Chem Biol., 2014, 10:613-622, Cox, A. D. et al., Nat Rev Drug Discov., 2014, 13:828-851). N-RAS and/or H-RAS mutations are frequently reported in colorectal cancer, bladder cancer, kidney cancer, thyroid carcinomas, melanoma, hepatocellular carcinoma, and hematologic malignancies (Cox, A. D. et al., Nat Rev Drug Discov., 2014, 13:828-851). Namely, Prior et al. found K-RAS as most frequent mutated isoform in analyzed tumors, namely in 22% of all tumors analyzed compared to about 8% for N-RAS and 3% for H-RAS (Prior, I. A. et al., Cancer Res., 2012, 72:2457-2467). In the majority of cases, these mutations are point mutations which introduce an amino acid substitution at position 12, 13, or 61 (Wang, Y. et al., J Med Chem., 2013, 56:5219-5230, Spiegel, J. et al., Nat Chem Biol., 2014, 10:613-622). Presence of said point mutations impairs GTPase activity, in particular renders RAS insensitive to GAP action with a resulting constitutive activation of RAS signaling pathways (Zimmermann, G. et al., J Med Chem., 2014, 57:5435-5448). Prior et al., for example, found that 80% of K-RAS mutations occur at codon 12, whereas very few mutations were observed at codon 61 or 13 (Prior, I. A. et al., Cancer Res., 2012, 72:2457-2467).

K-RAS mutations are reported to be present in more than 25% of non-small cell lung cancers (NSCLC) usually associated with unfavorable clinical outcomes, and they have been reported to occur frequently in patients with lung adenocarcinoma (20-30%). K-RAS mutations are comparable uncommon in lung squamous cell carcinoma (Cox, A. D. et al., Nat Rev Drug Discov., 2014, 13:828-851). Constitutive activation of K-RAS leads to persistent stimulation of signaling pathways that promote tumorigenesis, including the raf/MEK/ERK and PI3K/AKT/mTOR signaling cascades that are downstream to K-RAS.

In the absence of such activating RAS mutations, an increased RAS activity such as by overexpression or increased activation of growth signaling pathways has been reported in tumors, too (Wang, Y. et al., J Med Chem., 2013, 56:5219-5230).

Different approaches have been described for inhibiting RAS protein signaling pathways including strategies to influence the distribution of RAS in the cell such as inhibition of farnesylation of RAS or inhibition of RAS membrane interactions as well as to specifically address the signaling pathways or to inhibit RAS protein directly (e.g. Spiegel, J. et al., Nat Chem Biol., 2014, 10:613-622). Although RAS makes up the most frequently mutated oncogene family in human cancer and more than three decades of intensive effort has been spent in the past decade to provide RAS inhibitors, no effective pharmacological inhibitor of the RAS protein has reached the clinic, which makes RAS to an “undrugable” protein.

Recently, Zimmermann et al. described a specific approach aimed at disrupting K-RAS membrane association by inhibiting cGMP phosphodiesterase delta subunit (“PDEδ”), a protein that can assist in RAS protein intracellular trafficking, in particular bind to farnesyl moieties and regulate the trafficking of RAS proteins to plasma membranes, i.e. facilitate the intracellular RAS diffusion and enhance its trapping at the right compartment. They identified and characterized a small-molecule PDEδ inhibitor, named deltarasin that inhibited the K-RAS-PDEδ interaction and impaired K-RAS signaling. In addition, deltarasin also strongly suppressed the proliferation of human pancreatic ductal adenocarcinoma cells in vitro and in vivo (Zimmermann, G. et al., Nature, 2013, 497:638-642, Zimmermann, G. et al., J Med Chem, 2014, 57:5435-5448).

In view of the limited clinical applicability of the majority of the approaches described so far and in view of frequent resistance mechanisms, there remains a strong need for compounds suitable for treating cancer, in particular for those being suitable to sufficiently and specifically inhibit RAS signaling.

### SUMMARY OF THE INVENTION

The present invention relates in a first aspect to a method for treating or preventing a disease, in particular cancer such as lung cancer like NSCLC adenocarcinoma, in a subject, in particular a mammal having a RAS gene mutation such as a K-RAS gene mutation.

Said method comprises administering an effective amount of a compound having Formula (I) or a pharmaceutically acceptable salt, solvate or anhydrate thereof to the subject:

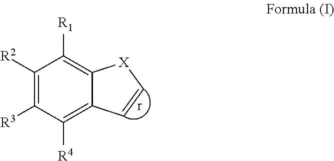

wherein:

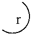

represents a 5- to 8-membered saturated, partially unsaturated or aromatic cyclic hydrocarbon;
X is selected from a N, S or O atom;
R¹, R² and R³ are each independently selected from hydrogen, straight chain or branched C₁-C₅-alkyl, —OH, —NH₂, straight chain or branched C₁-C₅-alkoxy or straight chain or branched C₁-C₅-alkylamino;
R⁴ is selected from —(CH₂)₂—R⁵, —(CH₂)₃—R⁵, —(CH₂)₄—R⁵, —CH₂—NH—R⁵, —(CH₂)₂—NH—R⁵, —(CH₂)₃—NH—R⁵, —CH═CH—NH—R⁵, —CH₂—CH═CH—NH—R⁵, —CH═CH—CH₂—NH—R⁵, —CH═CH—R⁵, —CH═CH—CH₂—R⁵, —CH═CH—(CH₂)₂—R⁵, —CH₂—CH═CH—CH₂—R⁵, —CH═N—NH—R⁵, —CH₂—CH═N—NH—R⁵, —CH═N—CH₂—NH—R⁵, —CH—NH—NH—R⁵, —(CH₂)₂—NH—NH—R⁵,

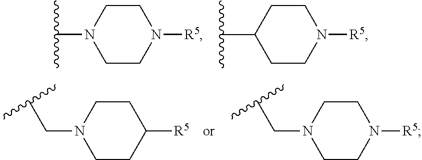

R⁵ is a moiety having a structure of Formula (II):

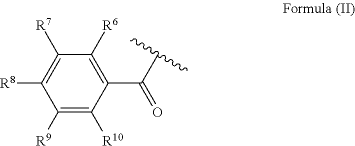

wherein R⁶, R⁷, R⁸, R⁹ and R¹⁰ are each independently selected from hydrogen, —OH, —NH₂, C₁-C₂-alkoxy or C₁-C₂-alkylamino, with the provisio that at least two of R⁶, R⁷, R⁸, R⁹ and R¹⁰ are each independently selected from —OH, —NH₂, C₁-C₂-alkoxy or C₁-C₂-alkylamino.

Hence, the compound of the present invention proved to comprise favorable geometric properties allowing for unexpected exceptional interacting with the PDEδ binding site and inhibition of PDEδ, in particular it comprises certain structural components, namely a three-cyclic backbone, i.e. the core part of the compound, a substituted benzoyl moiety in a side chain attached to the backbone having substituents including highly electronegative atoms, which benzoyl moiety is in particular attached to the three-cyclic backbone via a 2 to 4-membered aliphatic chain. It has been unexpectedly found that the presence of such structural components in the compound of Formula (I) allows for an advantageous interaction with amino acids in the prenyl-binding pocket of PDEδ and, thus, an exceptional inhibition of the interaction of PDEδ with RAS and respective RAS signaling pathways in cells by altering the localization of RAS proteins leading to an advantageous inhibiting of cancer cell proliferation and an apoptosis of the cells.

According to the invention is also the compound of Formula (I) for use as a medicament, preferably for use in the treatment of cancer such as lung cancer like NSCLC adenocarcinoma. Furthermore, the invention refers to the use of the compound of Formula (I) for preparing a medicament for treatment of a disease, in particular cancer such as lung cancer like NSCLC adenocarcinoma.

In another aspect, the present invention refers to a method for targeting cancer cells harboring a RAS gene mutation comprising the step of contacting said cells with a compound of Formula (I) or a salt, solvate or anhydrate thereof.

In particular, a method for inhibiting the proliferation of cancer cells is provided comprising the step of contacting cancer cells that include cells harboring a K-RAS gene mutation with an effective amount of the compound of Formula (I) or a salt, solvate or anhydrate thereof; and inhibiting the proliferation of the cells harboring a K-RAS gene mutation, wherein PDEδ is inhibited and proliferation of the cancer cells harboring a K-RAS gene mutation is selectively inhibited.

In still another aspect, the present invention provides a pharmaceutical composition comprising a compound of Formula (I) or a pharmaceutically acceptable salt, solvate or anhydrate thereof as active ingredient. The pharmaceutical composition further comprises physiologically tolerable excipients and may additionally contain further active ingredients, in particular therapeutic compounds for treating cancer such as lung cancer like NSCLC adenocarcinoma. The present invention also refers to the use of said pharmaceutical composition for inhibiting PDEδ, such as for inhibiting the signaling pathways downstream to RAS, in particular to K-RAS.

Other features and aspects of the invention will become apparent by consideration of the following detailed description and accompanying drawings.

### BRIEF DESCRIPTION OF THE DRAWINGS

FIG. 1A shows a 3D schematic representation of the structure of the compound of Formula (VII), the benzimidazole inhibitor and the interaction mode of the benzimidazole inhibitor with the binding pocket of the K-RAS protein. Hydrogen bonds formed by the benzimidazole are indicated.

FIG. 1B shows a 3D schematic representation of the binding mode and binding interactions between the compound of Formula (VII) and the binding pocket of K-RAS protein. Hydrogen bonds formed by the compound of Formula (VII) are indicated.

FIG. 2A shows the cell viability relative to untreated controls of A549 cells after treatment with the compound of Formula (VII) with a concentration of 0-10 μM for 72 h.

FIG. 2B shows the cell viability relative to untreated controls of H2122 cells after treatment with the compound of Formula (VII) with a concentration of 0-10 μM for 72 h.

FIG. 2C shows the cell viability relative to untreated controls of H358 cells after treatment with the compound of Formula (VII) with a concentration of 0-10 μM for 72 h.

FIGS. 3A, 3B, 3C, 3D, and 3E refer to a Flow Cytometry pattern of A549 cells having been treated with different concentrations of the compound of Formula (VII) namely with 2.5 μM (FIG. 3C), 5 μM (FIG. 3D) and 10 μM (FIG. 3E) compared with a Flow Cytometry pattern of A549 cells having been treated with 4 μM deltarasin (FIG. 3A) and a control group (FIG. 3B).

FIG. 3F is a bar chart showing the rate of apoptosis of A549 cells having been treated with the compound of Formula (VII) (referenced as “3237-1526”) with 2.5 μM, 5 μM or 10 μM or with 4 μM deltarasin and of the control group.

FIGS. 4A, 4B, 4C, 4D, 4E, and 4F show the formation of A549 cell colonies after treatment with different concentrations of the compound of Formula (VII), namely with 1.25 μM (FIG. 4C), 2.5 μM (FIG. 4D) and 5 μM (FIG. 4E) and 10 μM (FIG. 4F) compared with 4 μM deltarasin (FIG. 4A) and a control group (FIG. 4B).

FIG. 4G is a bar chart illustrating the average number of colonies formed in the colony formation assay as shown in FIG. 4A to 4F, i.e. with 1.25 μM, 2.5 μM, 5 μM and 10 μM of the compound of Formula (VII) (referenced as “3237-1526”) compared with 4 μM deltarasin and control group.

FIG. 5 refers to a western blot and shows the expression of p-C-raf, C-raf, pERK, ERK, pAKT and AKT of A549 cells treated with 4 μM deltarasin or 2.5 μM, 5 μM and 10 μM of the compound of Formula (VII) (referenced as “3237-1526”) compared to a control group.

FIG. 6 refers to an immunoblot pattern obtained after carrying out a K-RAS binding assay and indicates the amount of the active (GTP-bound) K-RAS of A549 cells treated with 4 μM deltarasin or 10 μM of the compound of Formula (VII) (referenced as “3237-1526”) compared to a control group.

### DESCRIPTION OF THE ILLUSTRATIVE EMBODIMENTS

The following embodiments and examples are given to enable those skilled in the art to more clearly understand and to practice the present invention. They should not be considered as limiting the scope of the invention, but merely as being illustrative and representing preferred embodiments thereof. The technical terms used in the present patent application have the meaning as commonly understood by a respective skilled person unless specifically defined otherwise.

The present invention refers in a first aspect to a method for treating or preventing, in particular for treating, a disease in a subject. Said method comprises administering an effective amount of a compound having Formula (I) or a pharmaceutically acceptable salt, solvate or anhydrate thereof to the subject:

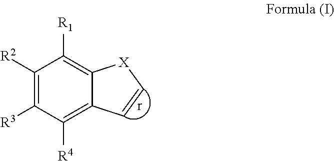

Said compound of the present invention is characterized by a three-cyclic ring structure, also referenced as three-cyclic backbone of the compound.

Namely, in the compound of Formula (I),

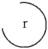

represents a 5- to 8-membered saturated, partially unsaturated or aromatic cyclic hydrocarbon. The term “cyclic hydrocarbon” refers to a hydrocarbon in which the carbon chain joins to itself in a ring, i.e. form a ring, namely the carbons are arranged in the form of a ring. 5- to 8-membered cyclic hydrocarbons include the saturated cyclic hydrocarbons cyclopentane, cyclohexane, cycloheptane or cyclooctane as well as partially unsaturated or aromatic derivates thereof. “Saturated” means that no double or triple bonds are formed in said cyclic hydrocarbon, wherein “unsaturated” refers to the presence of at least one double or triple bond in the cyclic hydrocarbon; i.e. in said embodiments, the cyclic hydrocarbon does not contain the maximum number of hydrogens. “Aromatic” means the presence of a delocalized, conjugated π-electron system, namely the term “aromatic” generally means a ring having a delocalized π-electron system containing 4n+2π electrons, where n is an integer and at least 0.

Preferably, the cyclic hydrocarbon is a 6- to 8-membered cyclic hydrocarbon, more preferably the cyclic hydrocarbon is a 6-membered cyclic hydrocarbon. Preferably, the cyclic hydrocarbon is partially unsaturated or saturated, most preferably the cyclic hydrocarbon is saturated, namely selected from cyclopentane, cyclohexane, cycloheptane or cyclooctane, in particular from cyclohexane, cycloheptane or cyclooctane. In an especially preferred embodiment, the cyclic hydrocarbon is a saturated 6-membered cyclic hydrocarbon, namely it is cyclohexane.

X in Formula (I) is selected from a N, S or O atom. Preferably, X is selected from a N or O atom, most preferably X is an O atom.

R¹, R² and R³ are generally selected from weakly to strongly electron-donating or activating groups, i.e. groups that donate some of their electron density into a conjugated system. R¹, R² and R³ are each independently selected from hydrogen, straight chain or branched C₁-C₅-alkyl, —OH, —NH₂, straight chain or branched C₁-C₅-alkoxy or straight chain or branched C₁-C₅-alkylamino.

The term “C₁-C₅ alkyl” as a group used in the present invention refers to a hydrocarbyl radical comprising from 1 to 5 carbon atoms. Accordingly, “C₁-C₄ alkyl” refers to a hydrocarbyl radical comprising from 1 to 4 carbon atoms and “C₃-C₄ alkyl” refers to a hydrocarbyl radical comprising from 3 to 4 carbon atoms. “Straight chain or branched C₁-C₅ alkyl” includes all linear or branched alkyl groups with 1 to 5 carbon atoms, and thus includes methyl, ethyl, n-propyl, isopropyl, butyl and its isomers (e.g. n-butyl, isobutyl, sec-butyl and tert-butyl), pentyl and its isomers (n-pentyl, tert-pentyl neopentyl, isopentyl, sec-pentyl, 3-pentyl). “Straight chain or branched C₃-C₄ alkyl” includes n-propyl, isopropyl, butyl and its isomers, namely n-butyl, isobutyl, sec-butyl and tert-butyl.

“Straight chain or branched C₁-C₅ alkoxy” refers to a radical having a formula -AB wherein A is an oxygen atom and B is a branched or straight chain C₁-C₅ alkyl, i.e. including, for example, methoxy, ethoxy, propyloxy, isopropyloxy, butyloxy and isobutyloxy. Accordingly, “straight chain or branched C₁-C₄ alkoxy” refers to a radical having a formula -AB wherein A is an oxygen atom and B is a branched or straight chain C₁-C₄ alkyl.

The term “alkylamine” refers to a radical having a formula —NBₓH_{y}, wherein x and y are selected from among x=1, y=1 and x=2, y=0. In a straight chain or branched C₁-C₅-alkylamine, B is a straight chain or branched C₁-C₅ alkyl, i.e. the number of carbon atoms in B is 1 to 5. When x=2, the total number of carbon atoms of both B groups is from 1 to 5, wherein the two B groups may contain a different number of carbon atoms provided that the total number of carbon atoms of both B groups is 1 to 5. “Straight chain or branched C₁-C₅ alkylamine” includes, for example, a N-methylamino-, N,N-dimethylamino-, N-ethylamino-, N,N-diethylamino- or N-propylamino-group. In a straight chain or branched C₁-C₄-alkylamine, B is a straight chain or branched C₁-C₄ alkyl.

Preferably, R¹, R² and R³ are each independently selected from hydrogen, straight chain or branched C₁-C₄-alkyl, —OH, —NH₂, straight chain or branched C₁-C₄-alkoxy or straight chain or branched C₁-C₄-alkylamino. More preferably, R¹, R² and R³ are each independently selected from hydrogen, straight chain or branched C₁-C₄-alkyl, —OH or —NH₂. In a more preferred embodiment of the present invention, R¹, R² and R³ are each independently selected from hydrogen, branched C₃-C₄-alkyl or —OH, in particular from hydrogen, branched C₄-alkyl or —OH, and still more preferably selected from hydrogen, tert-butyl or —OH. In especially preferred embodiments of the present invention, R¹ is hydrogen and R² and R³ are independently selected from branched C₃-C₄-alkyl or —OH, in particular independently selected from branched C₄-alkyl or —OH, more preferably independently selected from tert-butyl or —OH. Tert-butyl may also be referenced as 2-methylpropan-2-yl.

R⁴ represents a linking group, linking and connecting, respectively, the three-cyclic backbone of the compound of Formula (I) with R⁵, wherein R⁴ is in particular an aliphatic 2- to 4-membered linking group comprising atoms including carbon and preferably carbon and nitrogen atoms. R⁴ is selected from —(CH₂)₂—R⁵, —(CH₂)₃—R⁵, —(CH₂)₄—R⁵, —CH₂—NH—R⁵, —(CH₂)₂—NH—R⁵, —(CH₂)₃—NH—R⁵, —CH═CH—NH—R⁵, —CH₂—CH═CH—NH—R⁵, —CH═CH—CH₂—NH—R⁵, —CH═CH—R⁵, —CH═CH—CH₂—R⁵, —CH═CH—(CH₂)₂—R⁵, —CH₂—CH═CH—CH₂—R⁵, —CH═N—NH—R⁵, —CH₂—CH═N—NH—R⁵, —CH═N—CH₂—NH—R⁵, —CH₂—NH—NH—R⁵, —(CH₂)₂—NH—NH—R⁵,

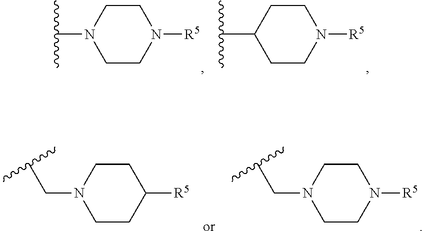

R⁴ is preferably selected from —(CH₂)₂—R⁵, —(CH₂)₃—R⁵, —(CH₂)₄—R⁵, —CH₂—NH—R⁵, —(CH₂)₂—NH—R⁵, —(CH₂)₃—NH—R⁵, —CH═CH—NH—R⁵, —CH₂—CH═CH—NH—R⁵, —CH═CH—CH₂—NH—R⁵, —CH═CH—R⁵, —CH═CH—CH₂—R⁵, —CH═CH—(CH₂)₂—R⁵, —CH₂—CH═CH—CH₂—R⁵, —CH═N—NH—R⁵, —CH₂—CH═N—NH—R⁵, —CH═N—CH₂—NH—R⁵, —CH₂—NH—NH—R⁵ or —(CH₂)₂—NH—NH—R⁵. More preferably, R⁴ is selected from —CH₂—NH—R⁵, —(CH₂)₂—NH—R⁵, —(CH₂)₃—NH—R⁵, —CH═CH—NH—R⁵, —CH₂—CH═CH—NH—R⁵, —CH═CH—CH₂—NH—R⁵, —CH═N—NH—R⁵, —CH₂—CH═N—NH—R⁵, —CH═N—CH₂—NH—R⁵, —CH₂—NH—NH—R⁵ or —(CH₂)₂—NH—NH—R⁵, i.e. R⁵ contains nitrogen atoms. In particular, R⁴ is selected from —CH═N—NH—R⁵, —CH₂—CH═N—NH—R⁵ or —CH═N—CH₂—NH—R⁵, further preferably R⁴ is —CH═N—NH—R⁵.

R⁵ is a substituted benzoyl with at least two substituents having a highly electronegative atom each, i.e. an atom with a high tendency to attract electrons or electron density towards itself, namely a N and/or O atom, i.e. having a high tendency to form hydrogen bonds with nearby hydrogen atoms such as in a protein, namely the latter proved to allow for the formation of advantageous hydrogen bonds in particular with Arg61 deep in the binding pocket of PDEδ and, additionally, with Trp90 and Leu38 in the prenyl-binding pocket of PDEδ. I.e. R⁴ is preferably a 2 to 4 membered linking group connecting said substituted benzoyl with the three-cyclic backbone of the compound of Formula (I), wherein said length of the linking group further supports the formation of advantageous hydrogen bonds in particular with the above mentioned amino acids and, thus, further contributes to an exceptional interaction with the prenyl-binding pocket of PDEδ.

R⁵ is a moiety of Formula (II):

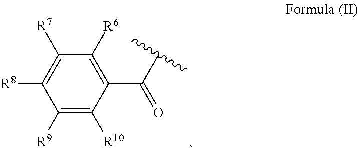

wherein R⁶, R⁷, R⁸, R⁹ and R¹⁰ are each independently selected from hydrogen, —OH, —NH₂, C₁-C₂-alkoxy or C₁-C₂-alkylamino, with the provisio that at least two of R⁶, R⁷, R⁸, R⁹ and R¹⁰ are independently selected from —OH, —NH₂, C₁-C₂-alkoxy or C₁-C₂-alkylamino.

Preferably, at least two of R⁶, R⁸, and R¹⁰ are independently selected from —OH, —NH₂, C₁-C₂-alkoxy or C₁-C₂-alkylamino, wherein the remaining R⁷ and R⁹ are hydrogen. More preferably, at least two of R⁶, R⁸ and R¹⁰ are independently selected from —OH or —NH₂, wherein the remaining R⁷ and R⁹ are hydrogen. Still more preferably, two of R⁶, R⁸, and R¹⁰ are each independently selected from —OH or —NH₂ with the third one and R⁷ and R⁹ being hydrogen. In particular, two of R⁶, R⁸, and R¹⁰ are selected from —OH and the third one and R⁷ and R⁹ are hydrogen. In especially preferred embodiments, R⁶ and R⁸ are —OH and R⁷, R⁹ and R¹⁰ are hydrogen.

The effective amount of the compound of Formula (I) may depend on the species, body weight, age and individual conditions of the subject and can be determined by standard procedures such as with cell cultures or experimental animals.

Said compound of Formula (I), amongst others, proved to comprise favorable geometric properties for interacting with the PDEδ prenyl-binding pocket, in particular structural components including a three-cyclic backbone such as a 1,2,3,4-tetrahydrodibenzo[b,d]furan, i.e. the core part of the compound, a substituted benzoyl moiety in a side chain attached to the backbone having at least two substituents including highly electronegative atoms, namely N and/or O atoms, in particular O atoms, which benzoyl moiety is, in particular, attached to the three-cyclic backbone via a 2 to 4-membered aliphatic chain comprising carbon atoms and/or heteroatoms, in particular comprising both of them. The inventors found that the presence of such structural components present in the compound of Formula (I) allows for an advantageous interaction with the PDEδ binding pocket, in particular with Arg61 deep in the binding pocket and Trp90 and Leu38 in the prenyl-binding pocket of PDEδ.

Further moieties which may be attached to the backbone or side chain according to Formula (I) do not impede the interaction of compound of Formula (I) with the prenyl-binding pocket of PDEδ and preferably allow for additional interactions including van der Waals forces and hydrogen bonds or hydrophobic interactions with the prenyl-binding pocket of PDEδ and, thus, further contribute to the exceptional interaction with PDEδ.

Hence, a compound having a structure of Formula (I) represents a highly promising opportunity in particular for treatment of patients such as with cancer, in particular those bearing a RAS-dependent, in particular a K-RAS-dependent cancer. Inhibition of PDEδ proved to be accompanied with deviated localization of RAS proteins and, thus, impaired RAS growth signaling pathway which affects tumor growth, namely reduces tumor growth. I.e. the compound of the present invention can be used for inhibiting, reducing or preventing the proliferation of cancer cells or inducing apoptosis of cancer cells. “RAS” as used in the present invention comprises N-RAS, H-RAS and K-RAS isoforms.

Also contemplated by the present invention are any pharmaceutically acceptable salts, hydrates, solvates, anhydrates as well as enantiomers and their mixtures, stereoisomeric forms, racemates, diastereomers and their mixtures of the compound of Formula (I).

As used herein, the term “solvate” refers to a complex of variable stoichiometry formed by a solute, i.e. compound of Formula (I), and a solvent. If the solvent is water, the solvate formed is a hydrate. As used herein, the term “anhydrate” means any compound free of the water of hydration, as would be understood in the art. Suitable pharmaceutically acceptable salts are those which are suitable to be administered to subjects, in particular mammals such as humans and can be prepared with sufficient purity and used to prepare a pharmaceutical formulation. The terms stereoisomers, diastereomers, enantiomers and racemates are known to the skilled person.

In preferred embodiments of the present invention, the compound is a compound of Formula (III):

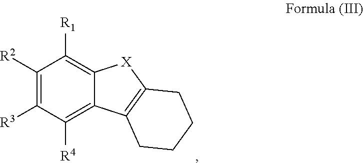

wherein:
X is selected from a N, S or O atom, preferably a N or O atom, most preferably an O atom;
R¹, R² and R³ are each independently selected from hydrogen, straight chain or branched C₁-C₄-alkyl, —OH, —NH₂, straight chain or branched C₁-C₄-alkoxy or straight chain or branched C₁-C₄-alkylamino, more preferably from hydrogen, straight chain or branched C₁-C₄-alkyl, —OH or —NH₂;
R⁴ is selected from —(CH₂)₂—R⁵, —(CH₂)₃—R⁵, —(CH₂)₄—R⁵, —CH₂—NH—R⁵, —(CH₂)₂—NH—R⁵, —(CH₂)₃—NH—R⁵, —CH═CH—NH—R⁵, —CH₂—CH═CH—NH—R⁵, —CH═CH—CH₂—NH—R⁵, —CH═CH—R⁵, —CH═CH—CH₂—R⁵, —CH═CH—(CH₂)₂—R⁵, —CH₂—CH═CH—CH₂—R⁵, —CH═N—NH—R⁵, —CH₂—CH═N—NH—R⁵, —CH═N—CH₂—NH—R⁵, —CH₂—NH—NH—R⁵ or —(CH₂)₂—NH—NH—R⁵, more preferably selected from —CH═N—NH—R⁵, —CH₂—CH═N—NH—R⁵ or —CH═N—CH₂—NH—R⁵;
R⁵ is a moiety of Formula (II), wherein R² and R⁹ are both hydrogen, i.e. having the Formula

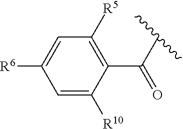

wherein R⁶, R⁸, and R¹⁰ are each independently selected from hydrogen, —OH, —NH₂, C₁-C₂-alkoxy or C₁-C₂-alkylamino, with the provisio that at least two of them are independently selected from —OH, —NH₂, C₁-C₂-alkoxy or C₁-C₂-alkylamino, preferably at least two of R⁶, R⁸, and R¹⁰ are independently selected from —OH or —NH₂.

In more preferred embodiments of the present invention, the compound is a compound of Formula (IV):

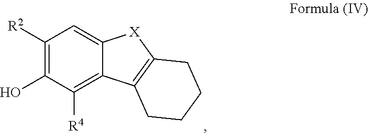

wherein:
X is selected from a N or O atom, most preferably an O atom;
R² is selected from straight chain or branched C₁-C₄-alkyl, —OH, —NH₂, straight chain or branched C₁-C₄-alkoxy or straight chain or branched C₁-C₄-alkylamino, more preferably from straight chain or branched C₁-C₄-alkyl, in particular from straight chain or branched C₃-C₄-alkyl;
R⁴ is selected from —CH₂—NH—R⁵, —(CH₂)₂—NH—R⁵, —(CH₂)₃—NH—R⁵, —CH═CH—NH—R⁵, —CH₂—CH═CH—NH—R⁵, —CH═CH—CH₂—NH—R⁵, —CH═N—NH—R⁵, —CH₂—CH═N—NH—R⁵, —CH═N—CH₂—NH—R⁵, —CH₂—NH—NH—R⁵ or —(CH₂)₂—NH—NH—R⁵, more preferably selected from —CH═N—NH—R⁵, —CH₂—CH═N—NH—R⁵ or —CH═N—CH₂—NH—R⁵;
R⁵ is a moiety of Formula (II), wherein R² and R⁹ are both hydrogen, i.e. having the Formula

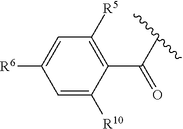

wherein R⁶, R⁸, and R¹⁰ are each independently selected from hydrogen, —OH, —NH₂, C₁-C₂-alkoxy or C₁-C₂-alkylamino, with the provisio that at least two of them are independently selected from —OH, —NH₂, C₁-C₂-alkoxy or C₁-C₂-alkylamino, preferably at least two of R⁶, R⁸, and R¹⁰ are each independently selected from —OH or —NH₂.

In further preferred embodiments of the present invention, the compound is a compound of Formula (V):

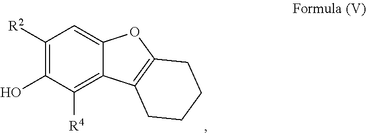

wherein:
R² is selected from straight chain or branched C₁-C₄-alkyl, —OH or —NH₂, more preferably from straight chain or branched C₁-C₄-alkyl, in particular from straight chain or branched C₃-C₄-alkyl;
R⁴ is selected from —CH═N—NH—R⁵, —CH₂—CH═N—NH—R⁵ or —CH═N—CH₂—NH—R⁵;
R⁵ is a moiety of Formula (II), wherein R² and R⁹ are both hydrogen, i.e. having the Formula

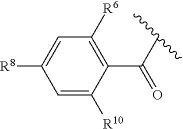

wherein R⁶, R⁸, and R¹⁰ are each independently selected from hydrogen, —OH, —NH₂, C₁-C₂-alkoxy or C₁-C₂-alkylamino, with the provisio that at least two of them are independently selected from —OH, —NH₂, C₁-C₂-alkoxy or C₁-C₂-alkylamino, preferably at least two of R⁶, R⁸, and R¹⁰ are independently selected from —OH or —NH₂.

In further preferred embodiments of the present invention, the compound has a structure of Formula (VI):

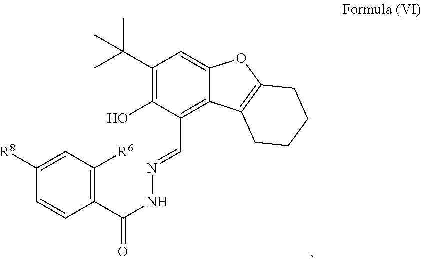

wherein R⁶ and R⁸ are each independently selected from —OH, —NH₂, C₁-C₂-alkoxy or C₁-C₂-alkylamino, preferably from —OH or —NH₂.

In especially preferred embodiments of the present invention, the compound has a structure of Formula (VII):

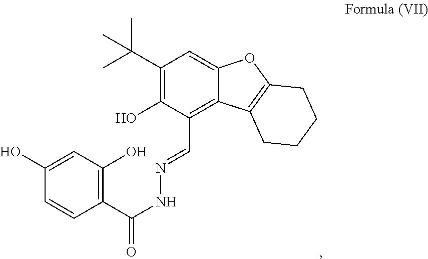 [SMILES: CC(C)(C)c1cc2oc3c(c2c(/C=N/NC(=O)c2ccc(O)cc2O)c1O)CCCC3]

which is also referenced as “3237-1526” herein and includes any pharmaceutically acceptable salts, hydrates, solvates, anhydrates as well as enantiomers and their mixtures, stereoisomeric forms, racemates, diastereomers and their mixtures of the compound of Formula (VII).

In particular, the method of the present invention refers to the treatment of the subject suffering from the disease. The subject is preferably a mammal, in particular a human. The disease is preferably cancer, in particular selected from pancreatic cancer, lung cancer, colorectal cancer, bladder cancer, kidney cancer, thyroid carcinomas, melanoma, hepatocellular carcinoma, and hematologic malignancies, preferably pancreatic cancer, lung cancer or colorectal cancer, further preferably lung cancer, more preferably NSCLC, still more preferably NSCLC of the adenocarcinoma type, i.e. NSCLC adenocarcinoma. NSCLC adenocarcinoma is the most common type in subjects who have never smoked and presence of NSCLC adenocarcinoma can be determined histologically. In a most preferred embodiment of the present invention, the disease is a K-RAS dependent NSCLC adenocarcinoma.

The terms “cancer” and “cancerous” refer to or describe a physiological condition in subjects in which a population of cells are characterized by unregulated cell growth. The term “tumor” simply refers to a mass being of benign (generally harmless) or malignant (cancerous) growth.

“RAS-dependent”, in particular “K-RAS-dependent” as used herein refers to a cancer or cancer cells having an enhanced expression or activity of a RAS protein such as K-RAS protein. This can be assessed by the activation of one or more downstream pathways to RAS such as to K-RAS. “Enhanced expression” or “Enhanced activity” preferably means an increase in RAS protein expression or RAS protein activity by at least 5% compared to a reference control, i.e. normal (healthy) cells, i.e. non-cancerous cells. In particular, the RAS protein is a RAS mutant protein, in particular a K-RAS mutant protein. The skilled person is able to determine the level of the expression of RAS such as K-RAS protein and/or the RAS such as K-RAS protein activity with common methods, for example, with well-known immunological assays that utilize antibody methods, Northern blotting, in-situ hybridization or similar techniques or qRT-PCR, RAS Activation Kits for determining the active form of RAS or by measuring the level of downstream effectors of the signaling pathway downstream to RAS.

In particular, said enhanced RAS expression or enhanced activity is essentially required for viability of the cells, i.e. the RAS protein expression or activity is highly correlated with the growth of the cancer cells and its inhibition results in a further enhanced growth suppression and cell death, i.e. the enhanced RAS such as K-RAS protein expression or activity is preferably the decisive factor essentially required for the survival of the cancer cells in RAS-dependent such as K-RAS-dependent cancers.

The subject in the method of the present invention preferably has at least one “RAS gene mutation”, i.e. at least one mutation such as translocation or transversion in the RAS protein encoding genes, i.e. in the respective nucleotide sequences, which in particular results in enhanced expression or enhanced activity of a “RAS mutant protein”. The expressed “RAS mutant protein”, in particular distinguishes from the wild-type RAS protein in the sequence of amino acids, especially at least one, in particular one amino acid has been replaced, also named substitution variant. “Wild type RAS protein” refers to a RAS protein with the sequence as present or encoded in normal (healthy) cells or tissue, namely non-cancerous cells or tissue, in particular without translocation or transversion in the RAS protein encoding genes.

RAS gene mutation is in particular accompanied by an aberrant function of the expressed RAS mutant protein favoring GTP binding and producing constitutive activation of RAS mutant protein with a resulting upregulation of signaling pathways thereby stimulating cell proliferation and inhibiting apoptosis and leading to uncontrolled cell growth. Preferably, the at least one RAS gene mutation concerns codons 12, 13 and/or 61 of the RAS protein encoding genes, more preferably, codon 12. In particular, the at least one RAS gene mutation is a K-RAS gene mutation at codons 12 and/or 13 in exon 2 and/or 61 in exon 3 of the K-RAS protein encoding gene, in particular at codons 12 or 13 in exon 2 or 61 in exon 3. The mutation is preferably accompanied by replacement of amino acids G12, G13 and/or Q61 in the active site of the RAS protein, in particular the K-RAS protein. I.e. the expressed RAS mutant protein, in particular K-RAS mutant protein, is preferably a protein which distinguishes from the RAS wild-type protein, in particular K-RAS wild-type protein with regards to amino acids G12, G13 and/or Q61, in particular one of them, further preferred with regards to G12.

In particular, the K-RAS gene mutation is a transversion mutation, i.e. a pyrimidine base is replaced with a purine base or vice versa, i.e. the K-RAS is accompanied by an amino acid substitution in the respective expressed K-RAS protein. In particular, the K-RAS gene mutation in the K-RAS protein encoding gene at codon 12 is selected from:
G12C (results in an amino acid substitution at position 12 in K-RAS protein, from a glycine (G) to a cysteine (C));
G12R (results in an amino acid substitution at position 12 in K-RAS, from a glycine (G) to an arginine (R));
G12S (results in an amino acid substitution at position 12 in K-RAS, from a glycine (G) to a serine (S));
G12A (results in an amino acid substitution at position 12 in K-RAS, from a glycine (G) to an alanine (A));
G12D (results in an amino acid substitution at position 12 in K-RAS, from a glycine (G) to an aspartic acid (D)); and/or
G12V (results in an amino acid substitution at position 12 in K-RAS, from a glycine (G) to a valine (V)).

The K-RAS gene mutation in the K-RAS protein encoding gene at codon 13 is preferably selected from:
G13C (results in an amino acid substitution at position 13 in K-RAS, from a glycine (G) to a cysteine (C));
G13R (results in an amino acid substitution at position 13 in K-RAS, from a glycine (G) to an arginine (R));
G13S (results in an amino acid substitution at position 13 in K-RAS, from a glycine (G) to a serine (S));
G13A (results in an amino acid substitution at position 13 in K-RAS, from a glycine (G) to an alanine (A)); and/or
G13D (results in an amino acid substitution at position 13 in K-RAS, from a glycine (G) to an aspartic acid (D)).

The K-RAS gene mutation in the K-RAS protein encoding genes at codon 61 is preferably selected from:
Q61K (results in an amino acid substitution at position 61 in K-RAS, from a glutamine (Q) to a lysine (K));
Q61L (results in an amino acid substitution at position 61 in K-RAS, from a glutamine (Q) to a leucine (L));
Q61R (results in an amino acid substitution at position 61 in K-RAS, from a glutamine (Q) to an arginine (R)); and/or
Q61H (results in an amino acid substitution at position 61 in K-RAS, from a glutamine (Q) to a histidine (H)).

I.e. the subject is preferably a mammal having at least one K-RAS gene mutation, wherein the K-RAS gene mutation is selected from a mutation in the K-RAS protein encoding gene at codons 12, 13 and/or 61 and is selected from G12C, G12R, G12S, G12A, G12D, G12V, G13C, G13R, G13S, G13A, G13D, Q61K, Q61L, Q61R and/or Q61H.

In particular, the subject is preferably a mammal having at least one K-RAS gene mutation at codon 12 in exon 2, more preferably one or more of, in particular one of G12C, G12A, G12D, G12S or G12V being the most frequent mutations in NSCLC adenocarcinoma.

Whether a subject has such RAS gene mutation can be detected with methods known to the skilled person such as DNA sequencing or commercially available test systems, DNA-DNA hybridization and the like.

The method of the present invention may further include steps carried out before administering the compound of Formula (I) to the subject comprising:

Obtaining a sample, in particular cancer or tumor cells from the subject; Testing said sample for the RAS expression levels, in particular the K-RAS expression levels, or identifying at least one RAS gene mutation, in particular K-RAS gene mutation such as selected from G12C, G12R, G12S, G12A, G12D, G12V, G13C, G13R, G13S, G13A, G13D, Q61K, Q61L, Q61R and/or Q61H; Optionally correlating the level of RAS expression, in particular K-RAS expression, with outcome and if conditions are met, administrating the compound of Formula (I) to said subject.

According to the invention is also the compound of Formula (I), in particular the compound of Formula (VII), for use as a medicament, preferably for use in the treatment of cancer such as lung cancer, especially NSCLC such as NSCLC adenocarcinoma, in particular RAS-dependent such as K-RAS-dependent NSCLC adenocarcinoma. The compound of Formula (I), in particular the compound of Formula (VII), can be used in an effective amount for treating a human. Another aspect of the invention refers to the use of the compound of Formula (I), in particular the compound of Formula (VII), for preparing a medicament for treatment of a disease, in particular of cancer, especially lung cancer, in particular NSCLC such as NSCLC adenocarcinoma, especially RAS-dependent such as K-RAS-dependent NSCLC adenocarcinoma.

The present invention provides in a further aspect a method for targeting cancer cells harboring a RAS gene mutation, in particular a K-RAS gene mutation, comprising the step of contacting said cells with a compound of Formula (I) or a salt, solvate or anhydrate thereof:

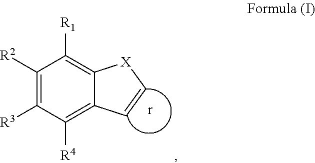

wherein

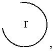

X and R¹ to R¹⁰ are as defined above including preferred embodiments as described above.

The compound of Formula (I) is preferably used in a concentration of at least 1.25 μM, more preferably at least 2.5 μM, more preferably at least 5 μM and in particular at least 10 μM. In particular, contacting said cells with the compound of Formula (I) leads to an inhibition, reduction or prevention of the proliferation of the cancer cells or induction of apoptosis of the cancer cells. The cancer cells are preferably contacted with the compound of Formula (I) for at least 10 h, more preferably for at least 12 hours.

The cancer cells are preferably from a lung tumor, in particular from a NSCLC, further preferred from a NSCLC adenocarcinoma. Preferably, the cancer is a RAS-dependent, in particular a K-RAS dependent cancer. Preferably, the RAS gene mutation is selected from a mutation in the RAS, in particular in the K-RAS, protein encoding genes at codons 12, 13 and/or 61, more preferably at codon 12. More preferably, the RAS gene mutation is a K-RAS gene mutation selected from G12C, G12R, G12S, G12A, G12D, G12V, G13C, G13R, G13S, G13A, G13D, Q61K, Q61L, Q61R and/or Q61H. More preferably, the K-RAS gene mutation is selected from one or more of G12C, G12A, G12D, G12S and G12V.

In preferred embodiments of the present invention, the compound is a compound of Formula (III):

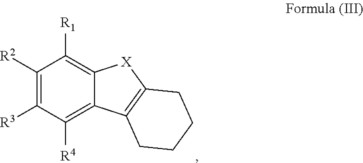

wherein:

X is selected from a N, S or O atom, preferably a N or O atom, most preferably an O atom; R¹, R² and R³ are each independently selected from hydrogen, straight chain or branched C₁-C₄-alkyl, —OH, —NH₂, straight chain or branched C₁-C₄-alkoxy or straight chain or branched C₁-C₄-alkylamino, more preferably from hydrogen, straight chain or branched C₁-C₄-alkyl, —OH or —NH₂; R⁴ is selected from —(CH₂)₂—R⁵, —(CH₂)₃—R⁵, —(CH₂)₄—R⁵, —CH₂—NH—R⁵, —(CH₂)₂—NH—R⁵, —(CH₂)₃—NH—R⁵, —CH═CH—NH—R⁵, —CH₂—CH═CH—NH—R⁵, —CH═CH—CH₂—NH—R⁵, —CH═CH—R⁵, —CH═CH—CH₂—R⁵, —CH═CH—(CH₂)₂—R⁵, —CH₂—CH═CH—CH₂—R⁵, —CH═N—NH—R⁵, —CH₂—CH═N—NH—R⁵, —CH═N—CH₂—NH—R⁵, —CH₂—NH—NH—R⁵ or —(CH₂)₂—NH—NH—R⁵, more preferably selected from —CH═N—NH—R⁵, —CH₂—CH═N—NH—R⁵ or —CH═N—CH₂—NH—R⁵; R⁵ is a moiety of Formula (II), wherein R² and R⁹ are both hydrogen, i.e. having the Formula

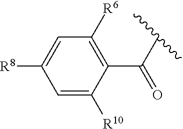

wherein R⁶, R⁸, and R¹⁰ are each independently selected from hydrogen, —OH, —NH₂, C₁-C₂-alkoxy or C₁-C₂-alkylamino, with the provisio that at least two of them are independently selected from —OH, —NH₂, C₁-C₂-alkoxy or C₁-C₂-alkylamino, preferably at least two of R⁶, R⁸, and R¹⁰ are independently selected from —OH or —NH₂.

In further preferred embodiments of the present invention, the compound is a compound of Formula (V):

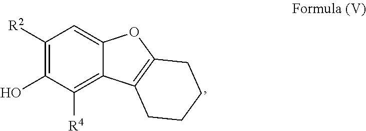

wherein:

R² is selected from straight chain or branched C₁-C₄-alkyl, —OH or —NH₂, more preferably from straight chain or branched C₁-C₄-alkyl, in particular from straight chain or branched C₃-C₄-alkyl; R⁴ is selected from —CH═N—NH—R⁵, —CH₂—CH═N—NH—R⁵ or —CH═N—CH₂—NH—R⁵; R⁵ is a moiety of Formula (II), wherein R² and R⁹ are both hydrogen, i.e. having the Formula

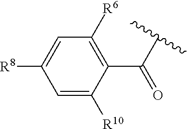

wherein R⁶, R⁸, and R¹⁰ are each independently selected from hydrogen, —OH, —NH₂, C₁-C₂-alkoxy or C₁-C₂-alkylamino, with the provisio that at least two of them are independently selected from —OH, —NH₂, C₁-C₂-alkoxy or C₁-C₂-alkylamino, preferably at least two of R⁶, R⁸, and R¹⁰ are independently selected from —OH or —NH₂.

In especially preferred embodiments of the present invention, the compound has a structure of Formula (VII):

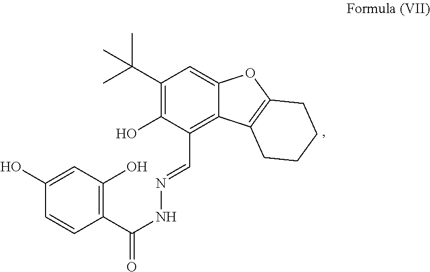 [SMILES: CC(C)(C)c1cc2oc3c(c2c(/C=N/NC(=O)c2ccc(O)cc2O)c1O)CCCC3]

wherein the concentration of the compound of Formula (VII) is at least 5 μM, preferably at least 10 μM.

In particular embodiments, the present invention refers to a method for inhibiting the proliferation of cancer cells comprising the step of contacting cancer cells that include cancer cells harboring a K-RAS gene mutation with an effective amount of the compound of Formula (I) or a salt, solvate or anhydrate thereof, in particular the compound of Formula (VII) or a salt, solvate or anhydrate thereof; and inhibiting the proliferation of the cells harboring a K-RAS gene mutation, wherein PDEδ is inhibited and proliferation of the cells harboring a K-RAS gene mutation is selectively inhibited.

In still another aspect, the present invention provides a pharmaceutical composition comprising a compound of Formula (I):

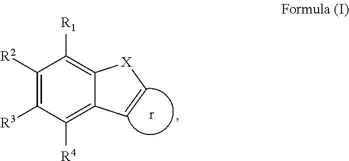

wherein

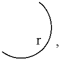

X and R¹ to R¹⁰ are as defined above including preferred embodiments as described above, or a pharmaceutically acceptable salt, solvate or anhydrate thereof as active ingredient and further comprising physiologically tolerable excipients.

Said pharmaceutical composition further comprises physiologically tolerable excipients. The skilled person is able to select suitable excipients depending on the form of the pharmaceutical composition and is aware of methods for manufacturing pharmaceutical compositions as well as able to select a suitable method for preparing the pharmaceutical composition depending on the kind of excipients and the form of the pharmaceutical composition.

The pharmaceutical composition according to the invention can be present in solid, semisolid or liquid form to be administered by an oral, rectal, topical, parenteral or transdermal or inhalative route to a subject, preferably a human.

The pharmaceutical composition may comprise further active ingredients, such as therapeutic compounds used for treating cancer, in particular lung cancer such as NSCLC, in particular NSCLC adenocarcinoma.

In preferred embodiments of the present invention, the compound is a compound of Formula (III):

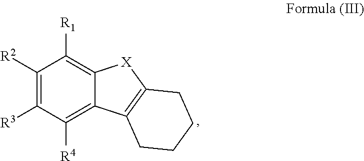

wherein:

X is selected from a N, S or O atom, preferably a N or O atom, most preferably an O atom; R¹, R² and R³ are each independently selected from hydrogen, straight chain or branched C₁-C₄-alkyl, —OH, —NH₂, straight chain or branched C₁-C₄-alkoxy or straight chain or branched C₁-C₄-alkylamino, more preferably from hydrogen, straight chain or branched C₁-C₄-alkyl, —OH or —NH₂; R⁴ is selected from —(CH₂)₂—R⁵, —(CH₂)₃—R⁵, —(CH₂)₄—R⁵, —CH₂—NH—R⁵, —(CH₂)₂—NH—R⁵, —(CH₂)₃—NH—R⁵, —CH═CH—NH—R⁵, —CH₂—CH═CH—NH—R⁵, —CH═CH—CH₂—NH—R⁵, —CH═CH—R⁵, —CH═CH—CH₂—R⁵, —CH═CH—(CH₂)₂—R⁵, —CH₂—CH═CH—CH₂—R⁵, —CH═N—NH—R⁵, —CH₂—CH═N—NH—R⁵, —CH═N—CH₂—NH—R⁵, —CH₂—NH—NH—R⁵ or —(CH₂)₂—NH—NH—R⁵, more preferably selected from —CH═N—NH—R⁵, —CH₂—CH═N—NH—R⁵ or —CH═N—CH₂—NH—R⁵; R⁵ is a moiety of Formula (II), wherein R² and R⁹ are both hydrogen, i.e. having the Formula

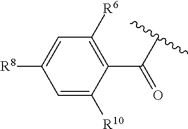

wherein R⁶, R⁸, and R¹⁰ are each independently selected from hydrogen, —OH, —NH₂, C₁-C₂-alkoxy or C₁-C₂-alkylamino, with the provisio that at least two of them are independently selected from —OH, —NH₂, C₁-C₂-alkoxy or C₁-C₂-alkylamino, preferably at least two of R⁶, R⁸, and R¹⁰ are independently selected from —OH or —NH₂.

In further preferred embodiments of the present invention, the compound is a compound of Formula (V):

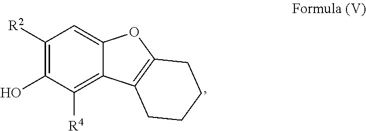

wherein:

R² is selected from straight chain or branched C₁-C₄-alkyl, —OH or —NH₂, more preferably from straight chain or branched C₁-C₄-alkyl, in particular from straight chain or branched C₃-C₄-alkyl; R⁴ is selected from —CH═N—NH—R⁵, —CH₂—CH═N—NH—R⁵ or —CH═N—CH₂—NH—R⁵; R⁵ is a moiety of Formula (II), wherein R² and R⁹ are both hydrogen, i.e. having the

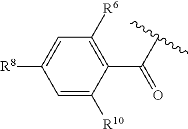

Formula wherein R⁶, R⁸, and R¹⁰ are each independently selected from hydrogen, —OH, —NH₂, C₁-C₂-alkoxy or C₁-C₂-alkylamino, with the provisio that at least two of them are independently selected from —OH, —NH₂, C₁-C₂-alkoxy or C₁-C₂-alkylamino, preferably at least two of R⁶, R⁸, and R¹⁰ are independently selected from —OH or —NH₂.

In especially preferred embodiments of the present invention, the compound has a structure of Formula (VII):

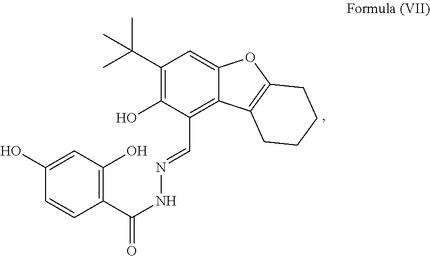 [SMILES: CC(C)(C)c1cc2oc3c(c2c(/C=N/NC(=O)c2ccc(O)cc2O)c1O)CCCC3]

or is any pharmaceutically acceptable salt, solvate or anhydrate thereof.

The present invention also refers to the use of the pharmaceutical formulation of the present invention for inhibiting PDEδ, especially for inhibiting the signaling pathways downstream to RAS mutant protein, in particular K-RAS mutant protein, in particular for reducing and suppressing, respectively, the phosphorylation of ERK, raf such as C-raf and AKT.

The skilled person is able to prepare the compound of Formula (I) with suitable purity and/or respective compounds are commercially available with sufficient purity.

### EXAMPLES

A549 (K-RAS^{G12S}), H358 (K-RAS^{G12C}), H2122 (K-RAS^{G12C}) and CCD19-Lu cells were obtained from the American Type Culture Collection and cultured in an environment of 5% CO₂ at 37° C. in RPMI-1640 medium supplemented with 10% fetal bovine serum (FBS), 100 units/mL penicillin, and 100 μg/mL streptomycin.

Deltarasin was purchased from Selleck Chemicals. Compound of Formula (VII), i.e. 3237-1526, was purchased from ChemDiv company. There were dissolved in DMSO to a 50 mM or 20 mM concentration and stored in small aliquots at −20° C. until further use. Antibodies to GAPDH, C-Raf, p-C-Raf p-AKT (Ser473), p-ERK (Thr202/Thy204) and ERK were purchased from Cell signaling Technology. Anti-AKT and K-RAS antibodies were acquired from Santa Cruz Biotechnology.

Descriptive analytical data are presented as means±SEM. Statistical analysis was conducted using Graph Prim5.0. One-way analysis of variance (ANOVA) was used to assess significant differences between datasets. Values of P<0.05 were considered statistically significant.

### Example 1

Firstly, the binding mode between the compound of Formula (VII) and K-RAS has been determined.

In this context, molecular docking calculation has been performed to study the interaction between the compound of Formula (VII) and PDEδ by Induced Fit Docking module in Schrodinger software (Schrodinger, Inc., New York, N.Y., 2009). The studied compound of Formula (VII) is prepared and optimized in the LigPrep module. The 3D structure of PDEδ in complex with a benzimidazole compound is derived from the PDB database (PDB ID: 4JV6) and prepared using the Protein Preparation Wizard. During the induced fit docking, centroid of the co-crystallized inhibitor was used to define the active site. The poses of the studied compound are evaluated by extra precision (XP) docking score and the conformation with the highest score is selected for binding mode analysis.

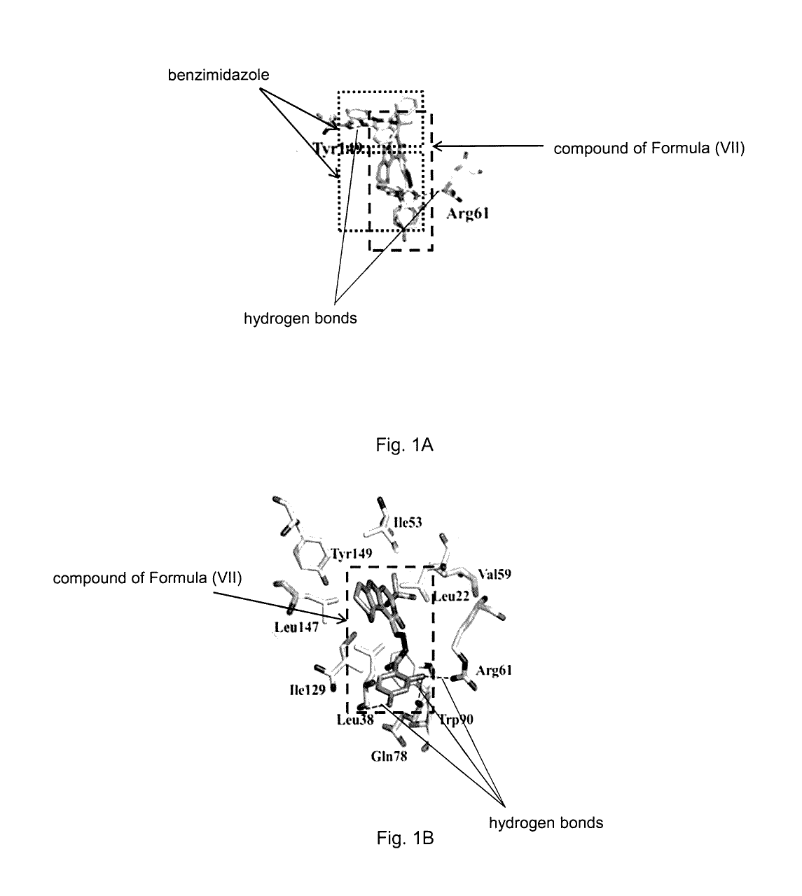

The binding affinity of compound of Formula (VII) to PDEδ was evaluated by the XP docking score. The docking score of compound of Formula (VII) is −13.270 Kcal/mol. The conformation of compound of Formula (VII) has been superimposed with the co-crystallized benzimidazole compound to compare their binding modes. As shown in FIG. 1A, the scaffold of compound of Formula (VII) overlapped well with the benzimidazole. As shown in FIG. 1B, the compound of Formula (VII) was buried in a hydrophobic pocket formed by Leu22, Leu38, Ile53, Val59, Arg61, Gln78, Trp90, Ile129, Leu147, Tyr149. Among these residues, Leu38, Arg61, Gln78 formed hydrogen bonds with the compound of Formula (VII).

### Example 2

In order to prove that the compound of Formula (VII) is highly cytotoxic and selective to cancer cells, the cytotoxic effect of the compound of Formula (VII) on lung cancer cell lines that have K-RAS gene mutation and normal lung epithelial cells (CCD19-Lu) has been determined.

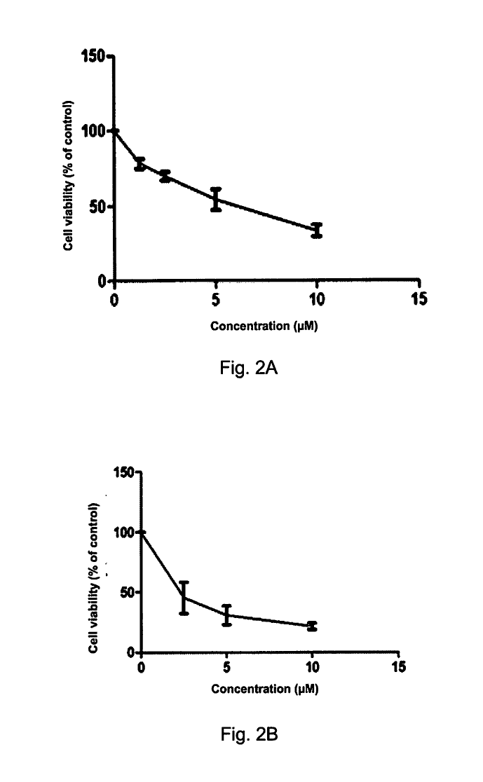

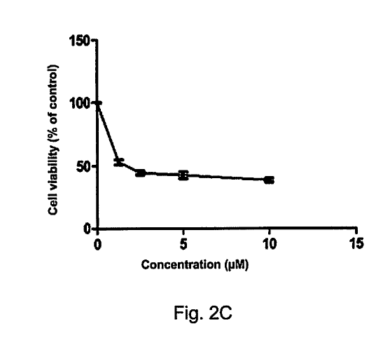

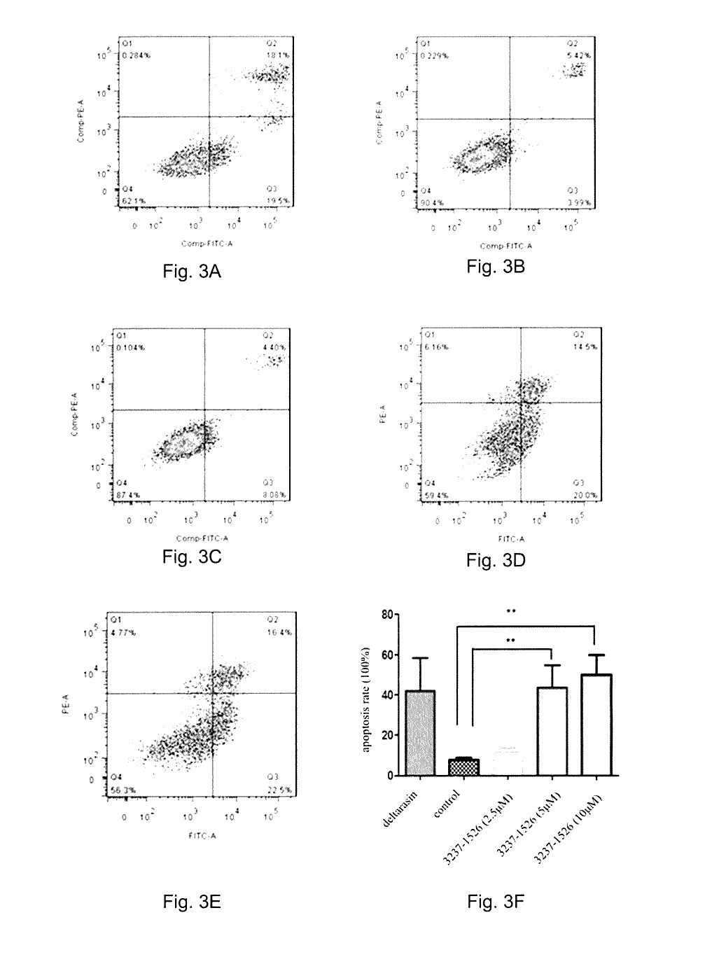

3000 cells were seeded on 96-well plates, cultured overnight for cell adhesion, then treated with DMSO or various concentrations of compound of Formula (VII) for 72 h, at the end of the incubation, each well was added with 10 μL of MTT (5 mg/mL; Sigma), and the plates were incubated for an additional 4 h, then the crystals were dissolved in 100 μL of the resolved solution (10% SDS and 0.1 mM HCL). The absorbance at 570 nm was measured using a microplate reader (Tecan, Morrisville, N.C., USA). The cell viability was calculated relative to untreated controls, with results based on at least three independent experiments. MTT assay showed that the antiproliferative effects of the compound of Formula (VII) in all cell lines with IC₅₀ of 5.59±1.27 μM, 2.4±2.1 μM and 3.35±2.74 μM for A549, H358 and H2122 cells, respectively (FIG. 2A to 2C), and it showed lower cytotoxicity in normal lung epithelial cells (CCD19-Lu). The IC₅₀ in CCD19-Lu is more than 20 μM (Table 1).

TABLE 1
IC₅₀ of the compound of Formula (VII) in different cell lines

| Cell lines | IC₅₀ (μM) |
|---|---|
| A549 | 5.59 ± 1.27 |
| H358 | 2.4 ± 2.1 |
| H2122 | 3.35 ± 2.74 |
| CCD-19 Lu | >20 |

### Example 3

Further, to provide additional evidence that the compound of Formula (VII) is potent and highly effective in inducing apoptosis in cancer cells, the induced apoptosis in A549 cells has been analyzed.

Apoptosis was measured using the Annexin V-FITC apoptosis detection kit (BD Biosciences, San Jose, Calif., USA), according to the manufacturer protocol. Briefly, A549 cells (1.0×105 cells/well) were allowed to attach in a 6-well plate for 24 h, cells were treated with the compound of Formula (VII) (2.5 μM, 5 μM or 10 μM) or 4 μM deltarasin for 48 h. Subsequently, cells were trypsinized, washed with PBS and stained with 100 μL binding buffer containing 2 μL Annexin-V FITC and 5 μL propidine iodide (PI) incubated in the dark at room temperature for 15 min, before further addition of 400 μL of 1× Annexin-binding buffer. The stained cells were analyzed quantitatively using a Flow Cytometer (BD Biosciences, San Jose, Calif., USA). Data were analyzed by Flow Jo software.

Flow cytometry analysis showed that the compound of Formula (VII) exhibited anti-cancer ability through induction of apoptosis on A549 cells in a concentration-dependent manner. Compared with the control group, treatment on A549 cells with the compound of Formula (VII) induced significant cell apoptosis as shown in FIG. 3A to 3F.

### Example 4

Still further, the inhibitory effect of the compound of Formula (VII) on the colony formation in A549 cells has been analyzed to provide further evidence that the compound of Formula (VII) inhibits the formation of colonies of cancerous cells to an exceptional degree, too. A549 cells were seeded on a six-well plate at a density of 500 cells per well. The cells were exposed to various concentrations of the compound of Formula (VII) (1.25 μM, 2.5 μM, 5 μM or 10 μM) or 4 μM deltarasin. After 10 days, the colonies were fixed with 4% paraformaldehyde and stained with a 0.5% (0.5% w/v) crystal violet solutions, the number of colonies >50 was counted under a dissecting microscope.

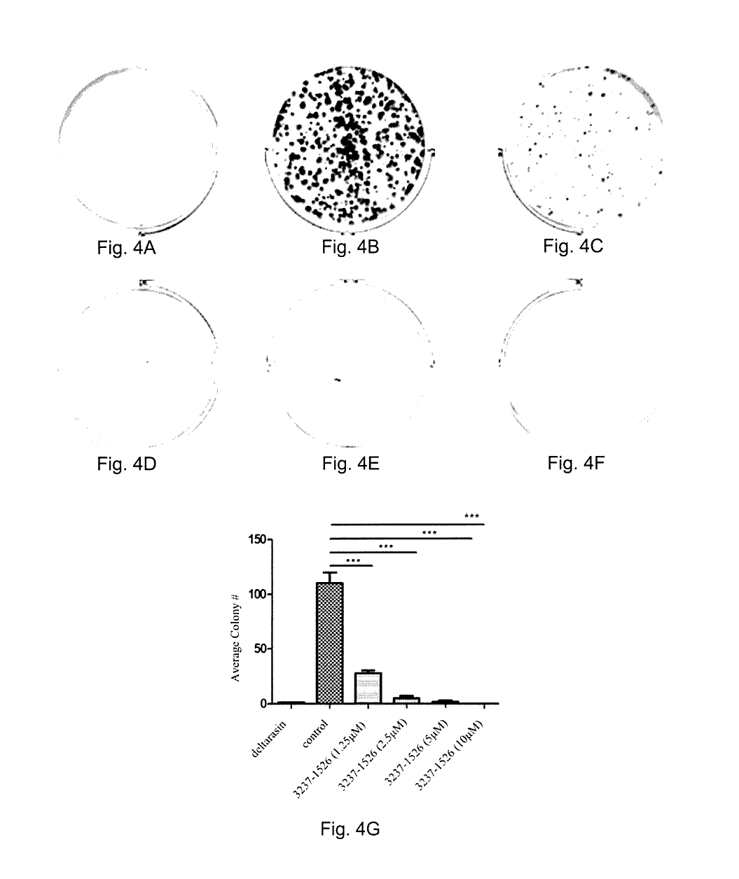

The analysis of the effect of the compound of Formula (VII) on colony formation activity revealed that the compound of Formula (VII) significantly inhibited the colony formation capacity of A549 (FIG. 4A to FIG. 4G). Notably, when the concentration of the compound of Formula (VII) reached 10 μM, A549 cells even formed no visible colonies.

### Example 5

Additionally, the suppression of the downstream signaling pathways to RAS by the compound of Formula (VII) has been tested.

Cells exposed to different concentrations of the compound of Formula (VII), namely 2.5 μM, 5 μM and 10 μM or 4 μM deltarasin as described above and a control group were washed twice with cold PBS then lysed in RIPA lysis buffer containing protease and phosphatase inhibitors, protein concentration of the cell lysates were measured using the Bio-Rad protein Assay kit (Bio-Rad, Philadelphia, Pa., USA). after equalizing the protein concentrations of the samples, 5× laemmli buffer was added and boiled at 100° C. for 5 min. Equal amounts of protein (20-40 μg per lane) were separated with a 10% SDS-PAGE gel, then the separated proteins were transferred to a Nitrocellulose (NC) membrane, which was then exposed to 5% non-fat dry milk in TBS containing 0.1% Tween 20 (0.1% TBST) for 1 hour at room temperature with constant agitation, followed by overnight incubation at 4° C. with primary antibodies, after washing three times by TBST, the membranes were incubated with secondary rabbit or mouse fluorescent antibodies, the signal intensity of the membranes was detected by anLI-COR Odessy scanner (Belfast, Me., USA). All primary antibodies were diluted 1:1000, while their recommended secondary antibodies were diluted 1:10000.

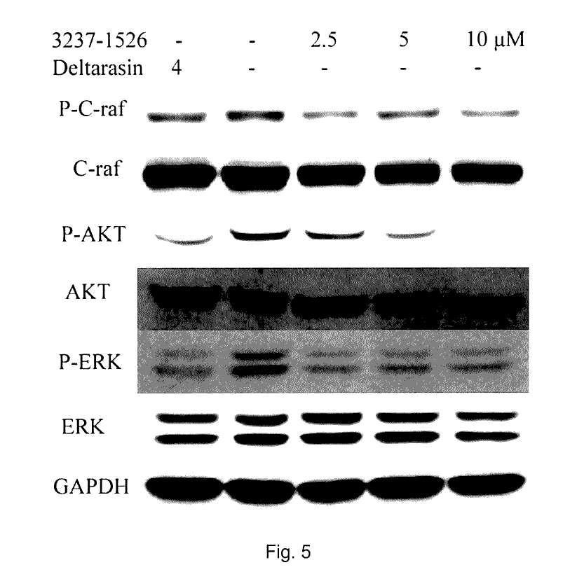

Treatment of A549 cells with the compound of Formula (VII) decreased the levels of p-C-raf, pErk and pAkt when compared with the untreated cells (FIG. 5), which proves that the compound of Formula (VII) is exceptionally suitable to suppress signaling pathways downstream to K-RAS.

### Example 6

Further, a K-RAS activation assay with subsequent immunoblotting has been carried out. A549 cells were treated with the compound of Formula (VII) for 48 h at 10 μM or deltarasin 4 μM. Cells were lysed in lysis buffer, adjusted the volume of each sample to 1 mL with 1× Assay Lysis Buffer, 40 μL of the Raf1 RBD Agarose bead slurry were added to each sample quickly, follow by incubating the tubes at 4° C. for 1 hour with gentle agitation, beads were washed three times with cold lysis buffer, and bounded protein was resuspended in 40 μL of 2× reducing SDS-PAGE sample buffer and heated at 100° C. for 5 min. The samples were then run by SDS-PAGE followed by immunoblotting. Total amount of RAS being pulled down were compared between the control and treatment groups.

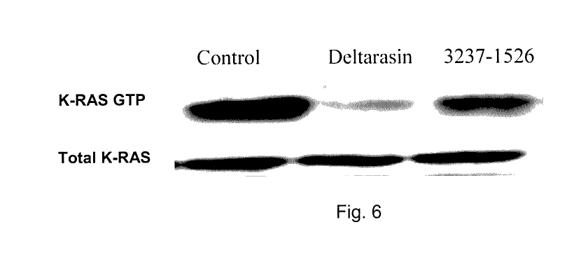

The compound of Formula (VII) inhibited K-RAS binding to GTP in A549 cells. By performing GTP pull down assay, treatment of A549 cells with the compound of Formula (VII) at 10 μM prior to probing with desthiobiotin-GTP, caused a decrease in the amount of K-RAS being pulled down with streptavidin as compared to the untreated control (FIG. 6). Deltarasin was used as positive control to demonstrate the suppression of GTP binding with K-RAS.

1. A method for treating non-small cell lung cancer adenocarcinoma in a subject comprising administering an effective amount of a compound having Formula (V) or a pharmaceutically acceptable salt, solvate or anhydrate thereof to the subject: 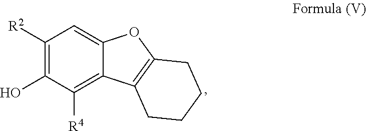
wherein:
R² is selected from straight chain or branched C₁-C₄-alkyl, —OH or —NH₂;
R⁴ is selected from —CH═N—NH—R⁵, —CH₂—CH═N—NH—R⁵ or —CH═N—CH₂—NH—R⁵;
R⁵ is a moiety having the Formula 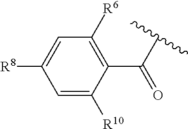
wherein R⁶, R⁸, and R¹⁰ are each independently selected from hydrogen, —OH, —NH₂, C₁-C₂-alkoxy or C₁-C₂-alkylamino, with the proviso that at least two of R⁶, R⁸, and R¹⁰ are independently selected from —OH, —NH₂, C₁-C₂-alkoxy or C₁-C₂-alkylamino.

2. (canceled)

3. (canceled)

4. The method of claim 1, wherein the NSCLC adenocarcinoma is K-RAS-dependent.

5. The method of claim 1, wherein the subject is a mammal having at least one RAS gene mutation and wherein the mutation concerns codons 12, 13 and/or 61 of the RAS encoding genes.

6. The method of claim 5, wherein the subject is a human having at least one K-RAS gene mutation and wherein the mutation concerns codon 12 of the K-RAS encoding gene and is selected from G12C, G12A, G12D, G12S and/or G12V.

7. (canceled)

8. (canceled)

9. The method of claim 1, wherein the compound is a compound having Formula (VII) or a pharmaceutically acceptable salt, solvate or anhydrate thereof: 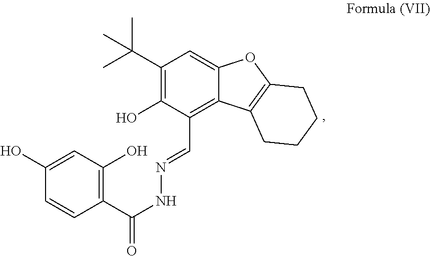 [SMILES: CC(C)(C)c1cc2oc3c(c2c(/C=N/NC(=O)c2ccc(O)cc2O)c1O)CCCC3]
and wherein the NSCLC adenocarcinoma is K-RAS-dependent.

10. A method for targeting cancer cells harboring a RAS gene mutation which are from a NSCLC adenocarcinoma, comprising the step of contacting said cells with a compound of Formula (V) or a salt, solvate or anhydrate thereof: 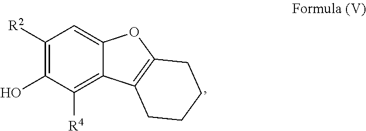
wherein:
R² is selected from straight chain or branched C₁-C₄-alkyl, —OH or —NH₂;
R⁴ is selected from —CH═N—NH—R⁵, —CH₂—CH═N—NH—R⁵ or —CH═N—CH₂—NH—R⁵;
R⁵ is a moiety having the Formula 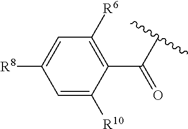
wherein R⁶, R⁸, and R¹⁰ are each independently selected from hydrogen, —OH, —NH₂, C₁-C₂-alkoxy or C₁-C₂-alkylamino, with the proviso that at least two of R⁶, R⁸, and R¹⁰ are independently selected from —OH, —NH₂, C₁-C₂-alkoxy or C₁-C₂-alkylamino.

11. The method of claim 10, wherein the proliferation of the cancer cells is inhibited, reduced or prevented or apoptosis of the cancer cells is induced.

12. (canceled)

13. (canceled)

14. The method of claim 10, wherein the compound of Formula (V) is used in a concentration of at least 2.5 μM.

15. The method of claim 10, wherein the compound is a compound having Formula (VII): 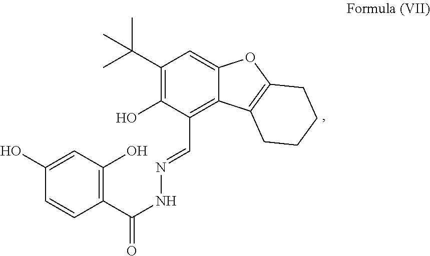 [SMILES: CC(C)(C)c1cc2oc3c(c2c(/C=N/NC(=O)c2ccc(O)cc2O)c1O)CCCC3]
and wherein the concentration of the compound of Formula (VII) is at least 5 μM.

16. The method of claim 10, wherein the cancer cells are contacted with the compound for at least 10 h.

17. The method of claim 10, wherein the cancer cells harbor at least one RAS gene mutation at codon 12 of the RAS protein encoding genes.

18. The method of claim 10, wherein the cancer cells harbor at least one K-RAS gene mutation at codon 12 of the K-RAS protein encoding gene selected from G12C, G12A, G12D, G12S and/or G12V.

19. (canceled)

20. (canceled)
